(19) United States
(12) Patent Application Publication
(10) Pub. No.: US 2023/0002778 A1
(43) Pub. Date: Jan. 5, 2023

(54) METHOD FOR IMPROVING GROWTH, STRESS TOLERANCE AND PRODUCTIVITY OF PLANT, AND INCREASING SEED QUALITY OF PLANT

(71) Applicants: ACADEMIA SINICA, Taipei City (TW); NATIONAL CENTRAL UNIVERSITY, Taoyuan City (TW)

(72) Inventors: Su-May YU, Taipei (TW); Yi-Shih CHEN, Yuanli Township (TW); Chung-An LU, Taipei City (TW); Tuan-Hua David HO, Taipei (TW)

(73) Assignees: ACADEMIA SINICA, Taipei City (TW); NATIONAL CENTRAL UNIVERSITY, Taoyuan City (TW)

(21) Appl. No.: 17/760,803

(22) PCT Filed: Sep. 17, 2020
(86) PCT No.: PCT/US2020/051247

§ 371 Date: Mar. 16, 2022

Related U.S. Application Data
(60) Provisional application No. 62/901,325, filed on Sep. 17, 2019.

(51) Int. Cl. C12N 15/82

(52) U.S. Cl. CPC C12N 15/8218; C12N 15/8273; C12N 15/8267

(57) ABSTRACT

Provided herein is a method for improving growth, stress tolerance and productivity of a plant. Also provided herein is a method for increasing seed quality of a plant. Specifically, the disclosure provides a method for improving growth, stress tolerance and productivity of a plant, comprising: providing a transgenic plant, which includes a reduced expression on an MYBS2 gene as relative to its wild-type counterpart; and a method for increasing seed quality of a plant, comprising: providing a seed from a transgenic plant, which overexpresses a full-length MYBS2 gene or a mutant MYBS2 gene as relative to its wild-type counterpart.

### BACKGROUND OF THE INVENTION

### 1. Field of the Invention

The present invention relates to a method for improving growth, stress tolerance and productivity of a plant. The present invention also relates to a method for increasing seed quality of a plant.

### 2. The Prior Art

Being autotrophic organisms, plants constantly monitor and respond to sugar status to maintain sugar homeostasis that is crucial for growth regulation, tolerance to environmental stresses and productivity. Mechanisms have evolved in plants in response to fluctuating sugar levels by adjusting metabolism to balance physiology. Sugar homeostasis, from the production of sugars in source tissues to their utilization or storage in sink tissues, is tightly coordinated through an integrated signaling network, involving crosstalk among sugars, hormones, and environmental cues, to regulate developmental and stress-adaptive processes. Sugars modulate nearly all fundamental processes throughout the entire lifecycle of plants. In general, sugar provision up-regulates genes involved in biosynthesis, transport, storage of reserves and cell growth, and it down-regulates those associated with photosynthesis, reserve mobilization and stress responses, but sugar starvation has the opposite effects.

Upon assimilation in photosynthetic source leaves, newly fixed carbon is utilized for cellular respiration and metabolism, transiently stored in vacuoles as sucrose or in plastids as starch, and transported as sucrose to sink tissues such as growing tissues (to generate energy) or developing organs (for long-term storage). Despite sugars being of central importance to plant growth, too much of them can be detrimental. For instance, ectopic expression of a yeast invertase, which converts sucrose to glucose and fructose in the apoplast, leads to decreased sucrose export and accumulation of carbohydrates in leaves, with subsequent inhibition of photosynthesis, stunted growth, impaired root formation, and necrosis in tobacco leaves. Rice and maize mutant lines defective in a tonoplast sucrose transporter, SUT2, accumulate higher concentrations of sugars in leaves but exhibit growth retardation and reduced biomass and grain yield, presumably due to reduced transport of sucrose out of vacuoles in source leaves to sink tissues/organs where sugar is in high demand.

Sugar starvation-induced nutrient recycling represents an essential strategy for survival or continuous growth under adverse environments. Plants undergo sugar starvation at certain times of their life cycles and in some of their non-green organs (such as roots, stems and flowers that do not carry out photosynthesis). Furthermore, sugar starvation or depletion can occur under extreme environmental conditions or upon attack by pathogens or pests that lead to significantly decreased photosynthetic efficiency, 110 or during a quiescent period or after leaf-shedding when photosynthesis is switched off. During prolonged darkness, sugar starvation drives a profound metabolic readjustment and initiates an autophagic pathway for partial degradation of chloroplast in vacuoles to recycle amino acids and other molecules that can be used to feed young tissues and produce seeds.

Starch, which constitutes approximately 75% of cereal grain dry weight, provides the major carbon source for generating energy and metabolites during germination and seedling growth. αAmy is the most abundant hydrolase and plays a central role in starch mobilization and thus the rate of seedling growth. Our previous studies in rice revealed that sugar starvation up-regulates αAmy expression by controlling its transcription rate and mRNA stability. αAmy transcriptional regulation is mediated through a sugar response complex (SRC) in αAmy promoters, in which the TA box is a key cis-acting element. MYBS1 is a single DNA binding repeat (R1) MYB transcription factor that interacts with the TA box and induces αAmy promoter under sugar starvation. Sugar starvation activates MYBS1 expression and promotes its nuclear import, whereas sugar provision has opposite effects.

GA activates αAmy promoters through the GA response complex (GARC), in which the adjacent GA response element (GARE) and TA box are key elements that act synergistically. The MYBS1-TA box interaction is essential for GARC and SRC functions, demonstrating that MYBS1 is a crucial node in GA and sugar starvation cross-signaling. In rice and barley, MYBGA is a GA-inducible R2R3 MYB transcriptional factor that binds the GARE and activates αAmy and hydrolase promoters in aleurone cells surrounding the starchy endosperm. GA antagonizes sugar-mediated repression of αAmy expression by enhancing co-nuclear transport of MYBGA and MYBS1 and formation of a stable bipartite MYB-DNA complex to activate αAmy and hydrolase gene promoters.

The 14-3-3 protein family is a highly conserved group of dimeric proteins that dock onto phosphorylated serine (Ser) and threonine (Thr) residues in their target proteins. In plants, target proteins of 14-3-3 proteins are involved in signal transduction and gene regulation of various biological processes, and binding of client proteins by 14-3-3 proteins may lead to conformational change, alternation of activity and stability, or sequestration in subcellular compartments. Involvement of 14-3-3 proteins in sugar regulation has been reported in yeast cells. In Saccharomyces cerevisiae, a 14-3-3 protein (Bmh1) is required for interaction with an HSP70 (Ssb) for recruiting a phosphatase (Glc7) to dephosphorylate and inactivate the protein kinase SNF1, a process necessary for glucose repression. In plants, 14-3-3 protein-mediated regulation of sugar homeostasis has not been explored.

Previously, we identified another R1 MYB transcription factor, MYBS2, which is up-regulated by sugars and also binds to the TA box of αAmy promoters. Here, by using αAmy as a biochemical marker, we showed that sugar provision/starvation counteract each other by regulating the competition between MYB S1 and MYB S2 for binding to the TA box as transcriptional activator and repressor of αAmy promoter, respectively. Particularly, phosphorylation regulates the sugar-dependent nucleocytoplasmic shuttling of MYBS2 and its cytoplasmic interaction with 14-3-3 proteins, which plays a critical role regulating the on/off switch of reversible gene expression to maintain sugar homeostasis. We also observed that manipulation of MYBS2 and αAmy can lead to beneficial effects on plant growth, tolerance to stress and grain productivity, which offers a novel approach to crop improvement.

### SUMMARY OF THE INVENTION

A primary objective of the present invention is to provide a method for improving growth, stress tolerance and productivity of a plant, comprising: (a) providing a transgenic plant, which includes a reduced expression on an MYBS2 gene as relative to its wild-type counterpart; and (b) growing the transgenic plant in a normal environment or an environment comprising an abiotic stress factor.

According to an embodiment of the present invention, the transgenic plant further includes an increased expression on an αAmy3 gene as relative to its wild-type counterpart.

According to an embodiment of the present invention, the MYBS2 gene encodes an MYBS2 transcription factor, and the MYBS2 transcription factor binds to a TA box in a promoter of the αAmy3 gene.

According to an embodiment of the present invention, the plant is a monocotyledonous plant or a dicotyledonous plant.

According to an embodiment of the present invention, the dicotyledonous plant is selected from the group consisting of Cucumis sativus, Ricinus communis, Solanum lycopersicum, Solanum tuberosum, Vitis vinifera, Populus trichocarpa, Arabidopsis thaliana, Arabidopsis lyrata, and Platycodon grandiflorus.

According to an embodiment of the present invention, the plant is a crop.

According to an embodiment of the present invention, the crop is rice, maize, wheat, barley, sugarcane, banana, cotton, soybean, pea, potato, tomato, brassica, orchid, balloon flower, yam, sweet potato, cassava, rose, petunia, chrysanthemum, lily, or carnation.

According to an embodiment of the present invention, the plant is an angiosperm.

According to an embodiment of the present invention, the MYBS2 gene encodes an MYBS2 transcription factor, and the MYBS2 transcription factor binds to a TA box in a promoter of the αAmy3 gene.

According to an embodiment of the present invention, the abiotic stress factor is osmotic stress, salt, dehydration, or heat.

Another objective of the present invention is to provide a method for increasing seed quality of a plant, comprising: (a) providing a seed from a transgenic plant, which overexpresses a full-length MYBS2 gene or a mutant MYBS2 gene as relative to its wild-type counterpart; and (b) growing the seed in a normal environment or an environment comprising an abiotic stress factor.

According to an embodiment of the present invention, the seed includes a reduced expression on an αAmy3 gene as relative to its wild-type counterpart.

According to an embodiment of the present invention, the mutant MYBS2 gene encodes a truncated MYBS2 transcription factor which includes deletion of 1st-53rd amino acid residues.

According to an embodiment of the present invention, the plant is a monocotyledonous plant or a dicotyledonous plant.

According to an embodiment of the present invention, the monocotyledonous plant is selected from the group consisting of Zea mays, Sorghum bicolor, Setaria italica, Hordeum vulgare, Brachypodium distachyon, Oryza sativa, Triticum spp., and Saccharum spp.

According to an embodiment of the present invention, the dicotyledonous plant is selected from the group consisting of Cucumis sativus, Ricinus communis, Solanum lycopersicum, Solanum tuberosum, Vitis vinifera, Populus trichocarpa, Arabidopsis thaliana, Arabidopsis lyrata, and Platycodon grandiflorus.

According to an embodiment of the present invention, the plant is a crop.

According to an embodiment of the present invention, the crop is rice, maize, wheat, barley, sugarcane, banana, cotton, soybean, pea, potato, tomato, brassica, orchid, balloon flower, yam, sweet potato, cassava, rose, petunia, chrysanthemum, lily, or carnation.

According to an embodiment of the present invention, the plant is an angiosperm.

According to an embodiment of the present invention, the abiotic stress factor is osmotic stress, salt, dehydration, or heat.

In summary, the present invention providing a method for improving growth, stress tolerance and productivity of a plant, and a method for increasing seed quality of a plant has the benefits on promoting plant growth and/or yield under normal or stressed conditions to cope with global water shortage in farmland and global climate warming, protecting plants from damages caused by water deficit, maintaining grain productivity under non-stressed and stressed conditions, maintaining high quality rice grain under global warming, enhancing public acceptance of new varieties and their products, reducing the transparency of milled rice to provide good sake-brewing rice for wine industry, enhancing transparency of milled rice for global market demand for high quality rice, maintaining the sugar homeostasis in plant, increasing sugar contents and stress tolerance, and generating new varieties to drought in various plant species.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1A is a data diagram demonstrating MYBS2 is a negative regulator of germination and plant growth, and suppresses αAmy expression, in which total mRNA were extracted from leaves of 7-day-old seedlings of transgenic lines overexpressing (Ox) or underexpressing (Ri) Ubi:MYBS2 and subjected to qRT-PCR analysis; the inset shows comparison of MYBS2 mRNA levels between sWT and Ri lines.

FIG. 1B is a data diagram demonstrating MYBS2 is a negative regulator of germination and plant growth, and suppresses αAmy expression, in which seeds were germinated in ½ MS medium without sugars at 28° C. for 5 days, before determining germination rates. Error bars represent standard deviation (SD).

FIG. 1C is a data diagram demonstrating MYBS2 is a negative regulator of germination and plant growth, and suppresses αAmy expression, in which two-day-old seedlings of sWT and MYBS2 Ox and Ri lines with similar shoot lengths were grown in ½ MS for up to 14 days; seedling growth was determined by measuring shoot length.

FIG. 1D is a data diagram demonstrating MYBS2 is a negative regulator of germination and plant growth, and suppresses αAmy expression, in which two-day-old seedlings of sWT and MYBS2 Ox and Ri lines with similar shoot lengths were grown in ½ MS for up to 14 days; seedling growth was determined by measuring shoot length.

FIG. 1E is a photograph demonstrating MYBS2 is a negative regulator of germination and plant growth, and suppresses αAmy expression, in which plants in FIG. 1C and FIG. 1D were transferred to a greenhouse for continuous growth and the morphology of 90-day-old plants was assessed.

FIG. 1F is a photograph demonstrating MYBS2 is a negative regulator of germination and plant growth, and suppresses αAmy expression, in which plants in FIG. 1C and FIG. 1D were transferred to a greenhouse for continuous growth and the morphology of 90-day-old plants was assessed.

FIG. 2A is a data diagram demonstrating MYBS2 represses αAmy promoter activities through the TA box, in which seedlings of sWT, MYBS2 (full-length or truncated cDNA) (XM_015757381.2) Ox and Ri lines were cultured in ½ MS medium without sugar for 10 days; total RNAs were extracted from leaves and used for qRT-PCR analysis using αAmy3-specific primers.

FIG. 2B is a schematic diagram demonstrating MYBS2 represses αAmy promoter activities through the TA box, in which rice embryo calli were co-transfected with effector (XM_015757381.2, AK068565) and reporter plasmids (see Chung-An Lu et al., (2002), Plant Cell, 14(8): 1963-1980), incubated in −S medium for 24 h, before assaying for luciferase activity. The value for luciferase activity of the reporter construct in the absence of the effector was set to 1×, and all other values were calculated relative to this value. Error bar indicates the standard error (SE) for three replicate experiments. Effector constructs in rice embryo calli carrying reporter constructs αAmy3-35Smp:Luc and 6×TA-35Smp:Luc (see Chung-An Lu et al., (2002), Plant Cell, 14(8): 1963-1980) in the presence of effector constructs.

FIG. 2C is a schematic diagram demonstrating MYBS2 represses αAmy promoter activities through the TA box, in which rice embryo calli were co-transfected with effector (XM_015757381.2, AK068565) and reporter plasmids (see Chung-An Lu et al., (2002), Plant Cell, 14(8): 1963-1980), incubated in −S medium for 24 h, before assaying for luciferase activity. The value for luciferase activity of the reporter construct in the absence of the effector was set to 1×, and all other values were calculated relative to this value. Error bar indicates the standard error (SE) for three replicate experiments. Luciferase activities of in rice embryo calli carrying reporter constructs αAmy3-35Smp:Luc and 6×TA-35Smp:Luc (see Chung-An Lu et al., (2002), Plant Cell, 14(8): 1963-1980) in the presence of effector constructs.

FIG. 3A is a data diagram demonstrating reduced MYBS2 expression upregulates αAmy3 under abiotic stress, and ectopic expression of αAmy3 enhances osmotic stress tolerance in rice, in which ten-day-old seedlings of sWT rice were treated with the indicated abiotic stress; total RNAs were extracted for qRT-PCR analysis using αAmy3- and MYBS2-specific primers; upper and lower panels show the expression of MYBS2 and αAmy3, respectively; CK: untreated control.

FIG. 3B is a data diagram demonstrating reduced MYBS2 expression upregulates αAmy3 under abiotic stress, and ectopic expression of αAmy3 enhances osmotic stress tolerance in rice, in which seeds of transgenic rice overexpressing Ubi: αAmy3 or αAmy3: αAmy3 were germinated in ½ MS medium without 400 mM sorbitol; germination rates were determined every day up to day 5; germination and plant growth without sorbitol treatment (−sorbitol).

FIG. 3C is a data diagram demonstrating reduced MYBS2 expression upregulates αAmy3 under abiotic stress, and ectopic expression of αAmy3 enhances osmotic stress tolerance in rice, in which seeds of transgenic rice overexpressing Ubi: αAmy3 or αAmy3: αAmy3 were germinated in ½ MS medium without 400 mM sorbitol; shoot length was determined at day 8; error bars represent SD (n=10); germination and plant growth without sorbitol treatment (−sorbitol).

FIG. 3D is a photograph demonstrating reduced MYBS2 expression upregulates αAmy3 under abiotic stress, and ectopic expression of αAmy3 enhances osmotic stress tolerance in rice, in which seeds of transgenic rice overexpressing Ubi: αAmy3 or αAmy3: αAmy3 were germinated in ½ MS medium without 400 mM sorbitol; plant morphology was photographed at day 8; germination and plant growth without sorbitol treatment (−sorbitol).

FIG. 3E is a data diagram demonstrating reduced MYBS2 expression upregulates αAmy3 under abiotic stress, and ectopic expression of αAmy3 enhances osmotic stress tolerance in rice, in which seeds of transgenic rice overexpressing Ubi: αAmy3 or αAmy3: αAmy3 were germinated in ½ MS medium with 400 mM sorbitol; germination rates were determined every day up to day 5; error bars represent SD (n=10); germination and plant growth with sorbitol treatment (+sorbitol).

FIG. 3F is a data diagram demonstrating reduced MYBS2 expression upregulates αAmy3 under abiotic stress, and ectopic expression of αAmy3 enhances osmotic stress tolerance in rice, in which seeds of transgenic rice overexpressing Ubi: αAmy3 or αAmy3: αAmy3 were germinated in ½ MS medium with 400 mM sorbitol; shoot length was determined at day 8; error bars represent SD (n=10); asterisks indicate significant differences (Student's t-test, *P<0.05, **P<0.01, ***P<0.001); germination and plant growth with sorbitol treatment (+sorbitol).

FIG. 3G is a photograph demonstrating reduced MYBS2 expression upregulates αAmy3 under abiotic stress, and ectopic expression of αAmy3 enhances osmotic stress tolerance in rice, in which seeds of transgenic rice overexpressing Ubi: αAmy3 or αAmy3: αAmy3 were germinated in ½ MS medium with 400 mM sorbitol; plant morphology was photographed at day 8; germination and plant growth with sorbitol treatment (+sorbitol).

FIG. 4A is a schematic diagram demonstrating reduced MYBS2 expression enhances osmotic and drought stress tolerance and grain yield in rice, in which ten-day-old seedlings were treated with 15% PEG for 7 days and 20% PEG for another 16 days before determining the survival rate; n=10; sWT, MYBS2-Ox, MYBS2(54-265)-Ox, and MYBS2-Ri lines were used.

FIG. 4B is a schematic diagram demonstrating reduced MYBS2 expression enhances drought stress tolerance in rice, in which ten-day-old seedlings were transferred to soil with regular water for 3 weeks, before cessation of watering for the next 21 days, and then the survival rate was determined; n=10; sWT, MYBS2-Ox, MYBS2(54-265)-Ox, and MYBS2-Ri lines were used.

FIG. 4C is a data diagram demonstrating reduced MYBS2 expression enhances grain yield in rice, in which grain yield of rice grown in field; n=12, significance levels with the t-test: *P<0.05, **P<0.01, ***P<0.001.

FIG. 4D is a data diagram demonstrating reduced MYBS2 expression enhances grain yield in rice in which grain yield of rice grown in field. *P<0.05, **P<0.01, ***P<0.001.

FIG. 4E is a data diagram demonstrating reduced MYBS2 expression enhances grain yield in rice in which grain yield of rice grown in greenhouse.**P<0.01, ***P<0.001.

FIG. 4F is a data diagram demonstrating reduced MYBS2 expression enhances grain yield in rice in which grain yield of rice grown in greenhouse. *P<0.05, **P<0.01, ***P<0.001.

FIG. 4G is a data diagram demonstrating reduced MYBS2 expression enhances grain yield in rice, wherein sWT, OX-1, OX-2, OX-3, OX-(tr)-1, OX-(tr)-2, RNAi-1, RNAi-2, and MYBS2-Cas9 (knock out) lines were used to analyze grain weight of rice in 2020 (field).

FIG. 5 is a schematic diagram demonstrating overexpression of MYBS2 reduces the chalky grains (i.e., increasing rice quality) while reduced expression of MYBS2 (or overexpression of αAmy3) increases the chalky grains.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

Sugar homeostasis is a unique feature in autotrophic plants where sugars are constantly generated and utilized. Plants have evolved mechanisms to cope with the fluctuation of sugar levels by adjusting metabolisms to balance physiological responses. The on/off switch of reversible gene expression by sugar starvation/provision represents a major mechanism for maintaining sugar homeostasis, however, details of the mechanism remain unclear. α-Amylase (αAmy) is the key enzyme hydrolyzing starch to provide sugars for plant growth, thus subject to sugar homeostasis regulation: induction by sugar starvation and repression by sugar provision. αAmy is induced by various stresses, but its physiological significance is also unclear. Here we discovered that the on/off switch of αAmy expression is regulated by two MYB transcription factors competing for the same promoter element; with MYBS1 promoting yet MYBS2 repressing αAmy expression. Sugar starvation promotes nuclear import of MYBS1 but nuclear export of MYBS2, and vice versa with sugar provision. Phosphorylation of MBYS2 at distinct serine residues plays critical roles in regulating its sugar-dependent nucleocytoplasmic shuttling and retaining by 14-3-3 proteins in the cytoplasm. Moreover, expression of MYBS2 is repressed by dehydration and heat, causing induction of αAmy3, yet activation of αAmy3 and suppression of MYBS2 leads to enhanced plant growth, stress tolerance and grain yield in rice. The present invention reveals unique insights into a critical regulatory mechanism for an on/off switch of reversible gene expression in maintaining sugar homeostasis, which tightly regulates plant growth and development, and also highlights MYBS2 and αAmy3 as potential targets for crop improvement.

Being autotrophic organisms, plants constantly monitor and respond to sugar status to maintain sugar homeostasis that is crucial for growth regulation, tolerance to environmental stresses and productivity. αAmy is the key enzyme hydrolyzing starch to sugars, thus subject to sugar homeostasis regulation: induction by sugar starvation but repression by sugar provision. Two MYBs compete for binding to the same promoter element and regulate this counteracting regulatory process, with MYBS1 promoting but MBYS2 repressing αAmy expression. Induction of αAmy expression by suppression of MYBS2 leads to enhanced stress tolerance and productivity. Phosphorylation of MBYS2 plays critical roles in regulating its sugar-dependent nucleocytoplasmic shuttling and interactions with 14-3-3 proteins, which constitutes a novel regulatory mechanism for reversible gene expression by sugar homeostasis.

During plant growth and development, sugar supply and starvation contrastingly regulate gene expression temporally and spatially, which play a pivotal role in determining crop productivity and quality. We identified MYBS2, a sugar inducible transcription factor that negatively regulates plant growth and development, by binding to the TA box and suppressing αAmy promoter activity. The MYBS2 promoter is specifically active in rice tissues rich in sugars. The present invention demonstrates that MYBS2 regulates sugar hemostasis in rice, and reveals that reduced MYBS2 expression leads to increased accumulation of α-amylase gene (αAmy3) expression, which in turn enhances plant growth, abiotic stress tolerance and grain yield in rice. The purpose of the present invention is to manipulate the expression of MYBS2 and αAmy3 to improve growth, abiotic stress tolerance, and grain yield in rice and other plant species.

### Definition

Numerical quantities given herein are approximate, and experimental values may vary within 20 percent, preferably within 10 percent, and most preferably within 5 percent. Thus, the terms “about” and “approximately” refer to within 20 percent, preferably within 10 percent, and most preferably within 5 percent of a given value or range.

### Materials and Methods

### Plant Materials

Rice (Oryza sativa cv Tainung 67) and barley (Hordeum vulgare cv Himalaya) were used in the present invention. Seedlings were grown in growth chambers under a 16-h light and 8-h dark cycle at 28° C.

Prior arts showed that sugar regulation of MYBS1 function in barley aleurone, SnRK1A (see Chung-An Lu et al., (2002), Plant Cell, 14(8): 1963-1980; Chung-An Lu et al., (2007), Plant Cell, 19(8): 2484-2499; Kuo-Wei Lee et al., (2009), Sci Signal, 2(91):ra61; Ya-Fang Hong et al., (2012), Plant Cell, 24(7):2857-73; Alan H. Christensen and Peter H. Quail, (1996), Transgenic Research, volume 5, pages 213-218) regulation of MYBS1 function using rice embryo calli, CIPK15 regulation of SnRK1A expression using rice suspension cells, regulation of MYBS1 and MYBGA interaction and nucleocytoplasmic shuttling of MYBS1 using rice and barley aleurones, and regulation of activity and nucleocytoplasmic shuttling of SnRK1A and MYBS1 using rice embryo calli and barley aleurones are all consistent, regardless of different systems being used. For our transient expression assay of luciferase activity, we chose to use rice embryo calli because they are easier to manipulate for large-scale sample preparation, particle bombardment and protein extraction.

### Primers

Primers for plasmid construction and RT-PCR analyses are listed in Table 1 and Table 2.

TABLE 1
Primers for RT-PCR and qRT-PCR analyses

| Primers | Nucleotide sequence (5′→3′) | Purpose |
|---|---|---|
| 3RT25A | GTAGGCAGGCTCTCTAGCCTCTAGG | αAmy3 RT-PCR |
| (F) | (SEQ ID NO: 1) | and real-time |
| 3RT(R) | AACCTGACATTATATATTGCACC | RT-PCR |
| | (SEQ ID NO: 2) | |
| 8RT1(F) | CTCAGGGTTCCTGCCGGTAGAAAGCA | αAmy 8 RT-PCR |
| | (SEQ ID NO: 3) | and real-time |
| 8RTB(R) | CGAAACGAACAGTAGCTAG | RT-PCR |
| | (SEQ ID NO: 4) | |
| S2QF | CAGACCAACCCTGGCAAAAA | MYBS2 real- |
| | (SEQ ID NO: 5) | time RT-PCR |
| S2QR | GAGGACTTGGAAGCTGATCATCA | |
| | (SEQ ID NO: 6) | |
| UBQ5(F) | ACCACTTCGACCGCCACTACT | RT-PCR and |
| | (SEQ ID NO: 7) | real-time |
| UBQ5(R) | ACGCCTAAGCCTGCTGGTT | RT-PCR |
| | (SEQ ID NO: 8) | |

TABLE 2
Primers for plasmid constructions and DNA sequence
mutation

| Primers | Nucleotide sequence (5′→3′) | Purpose |
|---|---|---|
| S2F Not1 | GCGCGGCCGCACCATGGAGCAGCATGAGGA | Ubi: |
| F | GGC (SEQ ID NO:9) | S2-Nos, |
| S2F BglII | GCAGATCTTCACAGCACCTTGATGGCCGCC | Ubi: |
| R | (SEQ ID NO: 10) | S2-Nos |
| tr S2Not1 | GCGCGGCCGCATGCCCAACCTCACCTCCA | Ub: |
| F | (SEQ ID NO: 11) | (tr)MYBS2- |
| tr S2bgl1 | GCAGATCTCAGCACCTTGATGGCCG | Nos, |
| R | (SEQ ID NO: 12) | |

### Plasmids

Plasmid p6XTA containing six copies of the fragment comprising positions −134 to −82 upstream of the transcription start site of αAmy3 (SEQ ID NO: 21) was fused to CaMV35S minimal promoter (SEQ ID NO: 18)-luciferase gene (Luc)(see Chung-An Lu et al., (2002), Plant Cell, 14(8): 1963-1980). Plasmid pUG containing the Ubiquitin (Ubi) promoter fused to the β-glucuronidase gene (GUS) was used as an internal control for transient expression assay. Plasmid p3Luc.18 contains αAmy3 SRC (−186 to −82 upstream of the transcription start site) fused to the CaMV35S minimal promoter-Adh1 intron-Luc cDNA fusion gene.

### Plasmid Construction

For overexpression of the full-length MYBS2 (SEQ ID NO: 13) and N-terminal-deleted MYBS2 (tr) in transgenic rice, MYBS2 cDNA fragments (AK121235) containing nucleotides 1-795 (full-length version) and nucleotides 160-795 (truncated version)(SEQ ID NO: 14) was inserted downstream of the Ubi promoter and upstream of the Nos terminator (SEQ ID NO: 19), generating pU-MYBS2 and pU-MYBS2(tr)(see Manuel Cercós et al., (2002), the plant journal, doi.org/10.1046/j.1365-313X.1999.00499.x). The two plasmids were digested with HindIII and inserted into the binary vector pCambia1301 (CAMBIA) at the same site, generating pAU-MYBS2 and pAU-MYBS2(tr)(AK121235). The amino sequence of the full length MYBS2 transcription factor is SEQ ID NO: 22.

For knockdown and knockout of MYBS2 in transgenic rice, we generated the MYBS2 RNA interference (RNAi) construct using a 485-bp (SEQ ID NO: 15) partial cDNA fragment of the MYBS2 3′UTR and the MYBS2 CRISPR-Cas9 construct using a 24 bp guide RNA sequence targeting MYBS2, respectively. For the MYBS2 RNA interference (RNAi) construct, the MYBS2 cDNA fragment was fused to the green fluorescent protein gene (GFP) and then inserted into the pSMY1H binary vector (AK121235); and for the knockout of MYBS2, the 24 bp MYBS2 guide RNA sequence was inserted into the pRGEB31 binary vector.

To generate common destination vectors for construction of plasmids used in our transient expression assays, we digested plasmid pAHC18 with BamHI to remove the Luc cDNA insert, before inserting the Not1, BglII and Spe1 restriction site sequences between the Ubi or CaMV35S promoter and the Nos terminator, generating destination vectors pUbi-Not1-BglII-SpeI-Nos.

### Rice Transformation

Plasmids pUbi-MYBS2, pUbi-MYBS2 RNAi, MYBS2-CAS9/pRGEB331 (SEQ ID NO: 16) and pUbi-MYBS2(tr)(see Alan H. Christensen and Peter H. Quail, (1996), Transgenic Research, volume 5, pages 213-218; ROBERT KAY et al., (1987), Science, Vol. 236, Issue 4806, pp. 1299-1302) were introduced into Agrobacterium tumefaciens strain EHA105 (Lifeasible, Cat #ACC-103), and rice (Tainung 67 (TNG67) transformation was performed.

### Rice Embryo Calli and Barley Aleurone Transient Expression Assays

Sample preparation and the particle bombardment transient expression assay using rice embryo calli was conducted. Error bars indicate the standard error for three replicate experiments. Plasmid pRS426, an unrelated yeast plasmid with a molecular mass similar to that of the effector plasmid, was used as the control plasmid.

### Real-Time Quantitative RT-PCR Analysis

Total RNA was extracted from tissues of rice seedlings with Trizol reagent (Invitrogen) and treated with RNase-free DNase I (Promega) at 37° C. for 30 min. One to five μg of RNA was used for cDNA synthesis using SuperScript™ III Reverse Transcriptase (Invitrogen), and cDNA was diluted 10-fold for storage. Two microliters of cDNA was mixed with 10 μM of primers and 10 μl of Power SYBR Green PCR Master Mix reagent (Applied Biosystems), and applied to an ABI 7500 Real-Time PCR system (Applied Biosystems). The rice UBQ5 gene was used as an internal control to normalize all data.

### Field Trial

To evaluate grain yield in the field, 25-day-old seedlings were transplanted to soil with 25×25 cm spacing between each plant. Seeds were harvested after ripening, dried and their yields were determined. Evaluations of grain yield in irrigated and non-irrigated fields were carried out. A total number of 26 plants of each line per plot were grown in the field, and only 12 plants in the middle were used to determine the grain yield.

### Statistical Analysis

In the present invention, n=30 for all experiments. Asterisks indicate significant differences (Student's t-test, *P<0.05, **<0.01, ***<0.001).

### Example 1

### MYBS2 is a Negative Regulator of Germination and Plant Growth, and Suppresses αAmy Expression

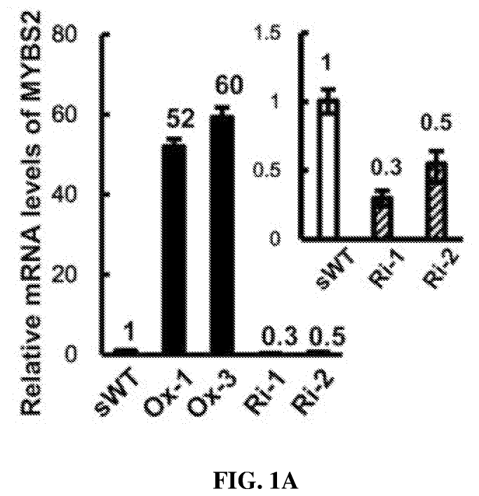

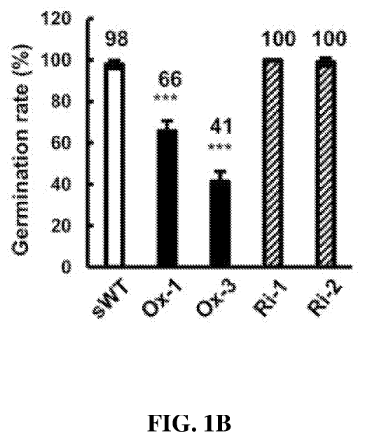

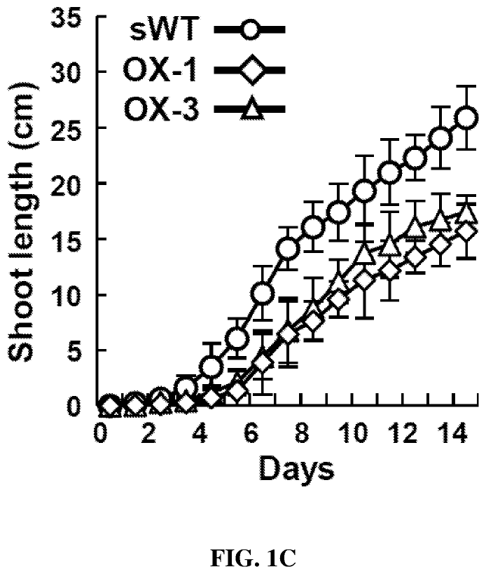

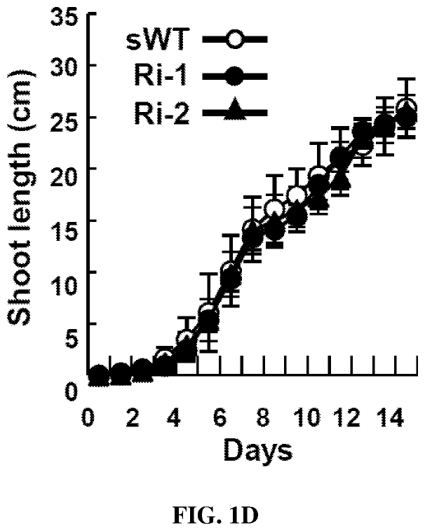

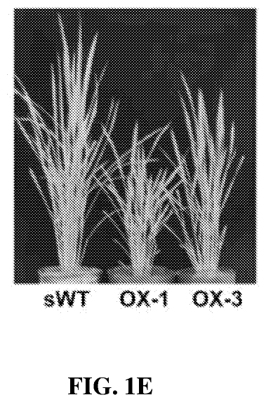

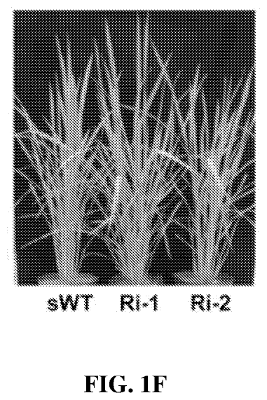

In this example, we first investigated the physiological function of MYBS2 in plant growth by gain- and loss-of-function analyses in transgenic rice overexpressing either MYBS2 cDNA or MYBS2 RNA interference (Ri) constructs under the control of the Ubi promoter (SEQ ID NO: 17). Levels of recombinant MYBS2 mRNAs increased by 52- to 60-fold in transgenic seedlings of two overexpressing (Ox) lines, whereas endogenous MYBS2 mRNA decreased by 50-70% in two silencing (Ri) lines, relative to the segregated wild type (sWT) (FIG. 1A). We compared the phenotypes of these transgenic lines to sWT and found that germination rates in two Ox lines were reduced by 34-59%, but were unchanged in the Ri lines (FIG. 1B). Moreover, seedling growth up to 14 days was delayed in Ox lines, but remained unchanged in the Ri lines (FIGS. 1C, 1D). Plant height at 90 days (i.e., directly before heading) was also shorter in Ox lines, but similar to sWT in Ri lines (FIGS. 1E, 1F).

### Example 2

### MYBS2 Represses αAmy Promoter Activities Through the TA Box

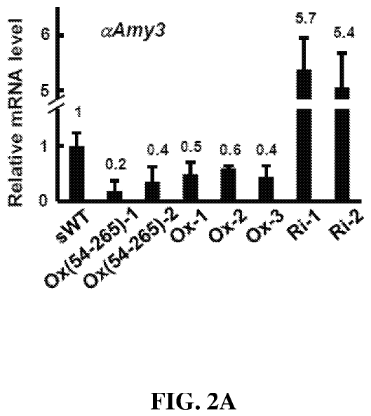

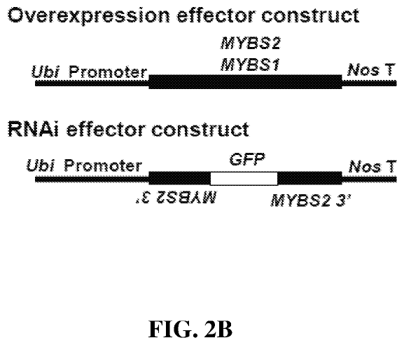

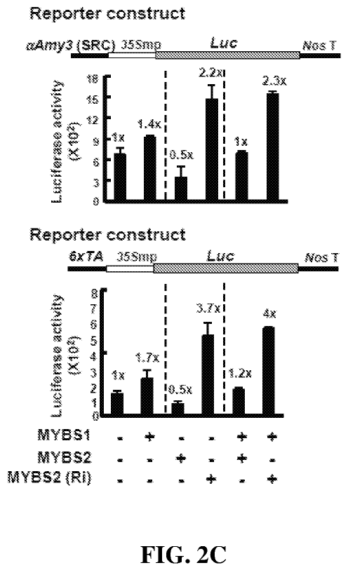

The expression of αAmy3 in rice seedlings was suppressed in MYBS2-Ox lines (by 40-60%), but was activated in MYBS2-RNAi lines (by 5- to 6-fold) (FIG. 2A). As shown in FIG. 2A, overexpression of MYBS2 (full-length or truncated cDNA) suppresses the expression of αAmy3, while reduction of MYBS2 increases the expression of αAmy3. Segregated wild (sWT), MYBS2-Ox, MYBS2(54-265)-Ox, and MYBS2-Ri lines were used in this example.

To understand the mechanism of MYBS2-mediated sugar repression of αAmy expression, we assessed the effect of MYBS1 (SEQ ID NO: 20), MYBS2 and MYBS2(Ri) (expression driven by the Ubi promoter) (FIG. 2B) on the activity of promoters containing the αAmy3 SRC and six tandem repeats of the TA box (6×TA) individually fused to the CaMV35S minimal promoter using a rice embryo transient expression system. Our results showed that the activity of the two promoters were enhanced by MYBS1, and even more significantly enhanced by the MYBS2 Ri construct, but were repressed by MYBS2 (FIG. 2C). These gain- and loss-of-function analyses suggest that MYBS2 is a transcriptional repressor and that it offsets the transcriptional trans-activation activity of MYBS1 on the TA box of αAmy3 SRC promoters.

Through transient expression assays, we found that αAmy3 and the 6×TA box promoters were suppressed (by 50%) by MYBS2 overexpression, and activated (by 2.2- to 3.7-fold) by MYBS2(Ri) (FIG. 2 C). This result is consistent with that in the transgenic stable expression assays, in which αAmy3 mRNA accumulation was suppressed (by 40-60%) by MYBS2 overexpression, and activated (by 5.4- to 5.7-fold) by MYBS2(Ri) (FIG. 2A).

### Example 3

### Reduced MYBS2 Expression Upregulates αAmy3 Under Abiotic Stress, and Ectopic Expression of αAmy3 Enhances Osmotic Stress Tolerance in Rice

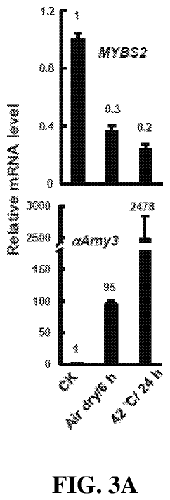

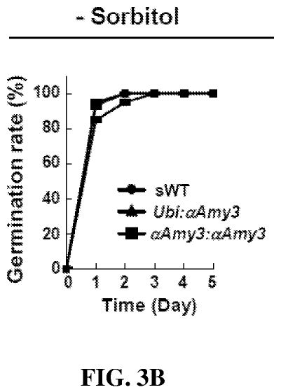

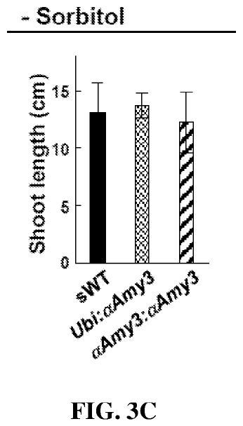

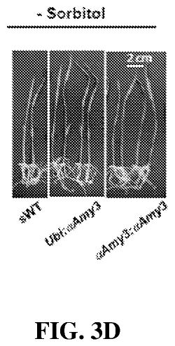

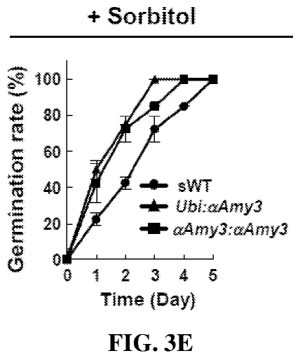

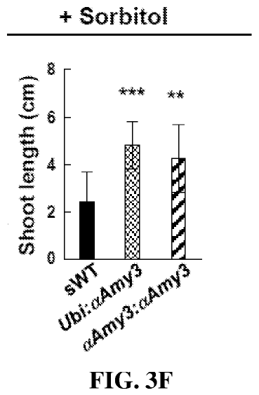

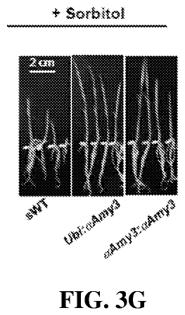

Since αAmy is activated by various biotic and abiotic stresses such as water stress, viral/bacterial infection, wounding, heat, or ABA in different plant species, we investigated whether MYBS2 regulates αAmy3 expression in response to abiotic stresses. We found that expression of MYBS2 in rice seedlings was suppressed by dehydration (air drying) (by 70%) and heat (42° C.) (by 80%), with the concomitant dramatic activation of αAmy3 expression under conditions of dehydration (by 95-fold) 390 and heat stress (by 2478-fold) (FIG. 3A).

To determine whether expression of αAmy3 is related to plant stress responses, we generated transgenic rice carrying Ubi: αAmy3 or αAmy3: αAmy3. We found that germination rates and shoot length of seedlings of all αAmy3-Ox lines were similar to sWT without sorbitol treatment (FIG. 3B-D) but were significantly greater than sWT under sorbitol treatment (i.e., mimicking osmotic stress) (FIG. 3E-G), indicating that ectopic expression of αAmy3 confers osmotic stress tolerance on rice.

In this example, we show that expression of MYBS2 is suppressed whereas that of αAmy3 is induced by dehydration and heat in rice seedlings (FIG. 3A). The results indicate that sugar production via αAmy-mediated starch degradation may be involved in stress tolerance in rice. This notion is supported by our analysis showing that ectopic overexpression of αAmy3 promotes the germination rate and seedling growth of rice under osmotic stress (FIGS. 3E-G).

### Example 4

### Reduced MYBS2 Expression Enhances Osmotic and Drought Stress Tolerance and Grain Yield in Rice

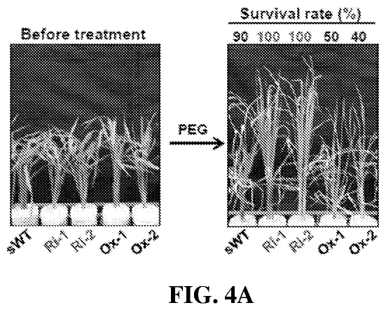

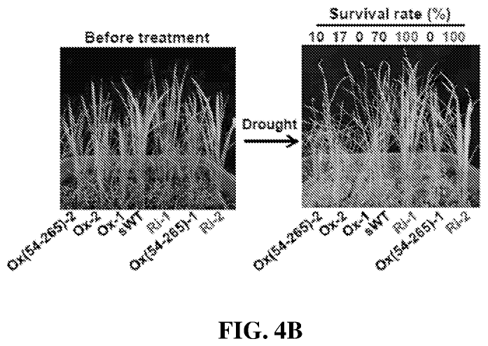

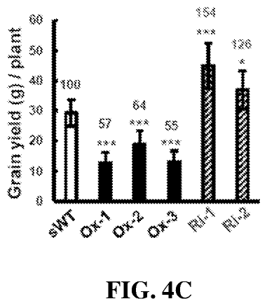

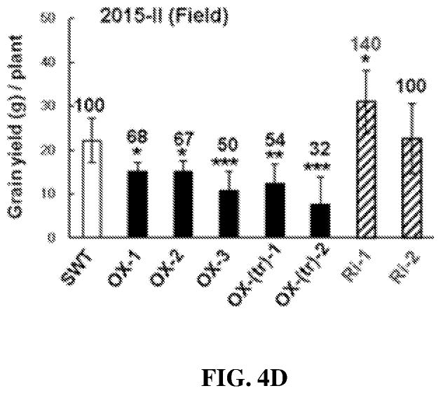

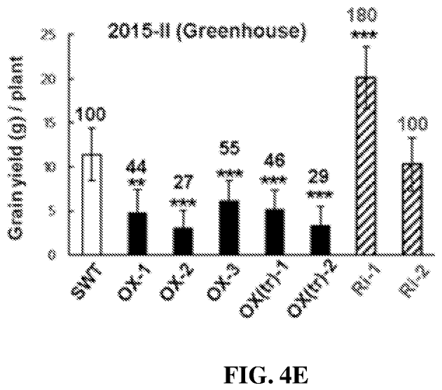

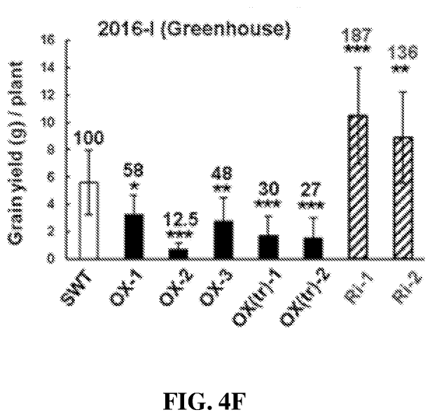

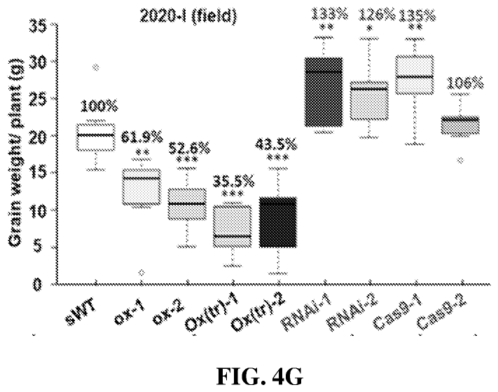

To further investigate whether down-regulation of MYBS2 improves plant stress tolerance, we subjected the MYBS2-Ox, MYBS2(54-265)-Ox and MYBS2-Ri lines to abiotic stress treatments. Using PEG treatment to mimic osmotic stress, we observed that the survival rate of the MYBS2-Ri lines (100%) were twice that of the MYBS2-Ox lines (40-50%) and higher than that for the sWT (90%) (FIG. 4A). Upon drought treatment in soil, the survival rate of the MYBS2-Ri lines (100%) was significantly greater than that of the sWT line (70%), and dramatically greater than that of the MYBS2-Ox lines (0-17%) and MYBS2(54-265)-Ox lines (0-10%) (FIG. 4B). Deletion of the N-terminal amino acid residues 1-53 caused exclusively nuclear localization of MYBS2, which is consistent with the very low expression of αAmy3 in rice seedlings (FIG. 2A) and the reduced survival rate of seedlings under drought stress (FIG. 4B). Since we had found that MYBS2 impairs seedling and plant growth (FIGS. 1C, 1E), we also evaluated the impact of MYBS2 expression on grain yield in field trials. These lines were grown in irrigated open field (FIG. 4D) or greenhouse in Spring (February to June) (FIG. 4E) or Fall (July to November)(FIG. 4F) season. We observed that grain yields were reduced by 40-70% for the MYBS2-Ox lines, but increased by 26-54% in the MYBS2-Ri lines, relative to the sWT (FIG. 4C). In addition, relative to the sWT, grain weight was increased by 106-135% in the MYBS2-Ri lines (i.e., RNAi-1 and RNAi-2 in FIG. 4G) and the MYBS2-Cas9 (knock out) lines (i.e., Cas9-1 and Cas9-2 in FIG. 4G). Together, these findings demonstrate that MYBS2 is a negative regulator of abiotic stress tolerance and grain yield.

Although reduced expression of MYBS2 does not affect plant growth under normal growth conditions (FIGS. 1D, 1F), it does promote osmotic and drought tolerance in transgenic rice plants, whereas overexpression of MYBS2 has the opposite effect (FIGS. 4A, 4B).

### Example 5

Overexpression of MYBS2 Reduces the Chalky Grains while Reduced Expression of MYBS2 (or Overexpression of αAmy3) Increases the Chalky Grains

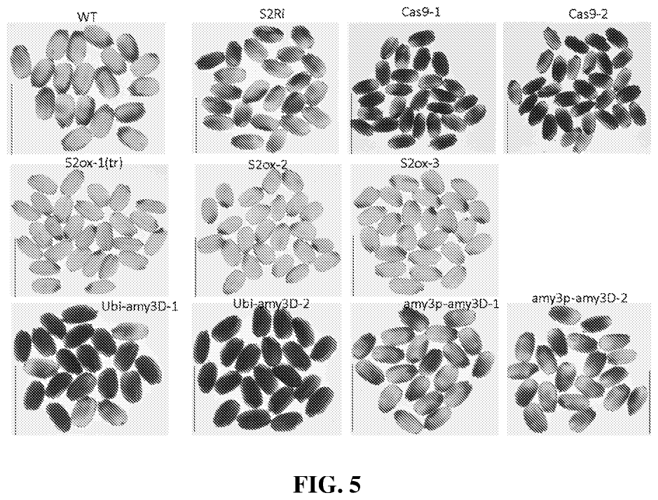

We also evaluated the impact of MYBS2 expression and its downstream target αAmy3 expression on grain (obtained from Example 4) quality. Our recent studies indicated that overexpression of MYBS2 can improve the seed quality by producing less-chalky seeds. In contrast, reduced expression of MYBS2 or increased expression of αAMY3 produced chalky grain (FIG. 5, in which ox means MYBS2 overexpression lines; Ubi-Amy3 means using Ubi as the promoter; Amy3p-αAmy3 means using the promoter of αAmy3D).

In summary, the present invention providing a method for improving growth, stress tolerance and productivity of a plant, and a method for increasing seed quality of a plant has the benefits on promoting plant growth and/or yield under normal or stressed conditions to cope with global water shortage in farmland and global climate warming, protecting plants from damages caused by water deficit, maintaining grain productivity under non-stressed and stressed conditions, maintaining high quality rice grain under global warming, enhancing public acceptance of new varieties and their products, reducing the transparency of milled rice to provide good sake-brewing rice for wine industry, enhancing transparency of milled rice for global market demand for high quality rice, maintaining the sugar homeostasis in plant, increasing sugar contents and stress tolerance, and generating new varieties to drought in various plant species.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for improving growth, stress tolerance and productivity of a plant, comprising:
(a) providing a transgenic plant, which includes a reduced expression on an MYBS2 gene as relative to its wild-type counterpart; and
(b) growing the transgenic plant in a normal environment or an environment comprising an abiotic stress factor.

2. The method according to claim 1, wherein the transgenic plant further includes an increased expression on an αAmy3 gene as relative to its wild-type counterpart.

3. The method according to claim 2, wherein the MYBS2 gene encodes an MYBS2 transcription factor, and the MYBS2 transcription factor binds to a TA box in a promoter of the αAmy3 gene.

4. The method according to claim 1, wherein the plant is a monocotyledonous plant or a dicotyledonous plant.

5. The method according to claim 4, wherein the monocotyledonous plant is selected from the group consisting of Zea mays, Sorghum bicolor, Setaria italica, Hordeum vulgare, Brachypodium distachyon, Oryza sativa, Triticum spp., and Saccharum spp.

6. The method according to claim 4, wherein the dicotyledonous plant is selected from the group consisting of Cucumis sativus, Ricinus communis, Solanum lycopersicum, Solanum tuberosum, Vitis vinifera, Populus trichocarpa, Arabidopsis thaliana, Arabidopsis lyrata, and Platycodon grandiflorus.

7. The method according to claim 1, wherein the plant is a crop.

8. The method according to claim 7, wherein the crop is rice, maize, wheat, barley, sugarcane, banana, cotton, soybean, pea, potato, tomato, brassica, orchid, balloon flower, yam, sweet potato, cassava, rose, petunia, chrysanthemum, lily, or carnation.

9. The method according to claim 1, wherein the plant is an angiosperm.

10. The method according to claim 1, wherein the abiotic stress factor is osmotic stress, salt, dehydration, or heat.

11. A method for increasing seed quality of a plant, comprising:
(a) providing a seed from a transgenic plant, which overexpresses a full-length MYBS2 gene or a mutant MYBS2 gene as relative to its wild-type counterpart; and
(b) growing the seed in a normal environment or an environment comprising an abiotic stress factor.

12. The method according to claim 11, wherein the seed includes a reduced expression on an αAmy3 gene as relative to its wild-type counterpart.

13. The method according to claim 11, wherein the mutant MYBS2 gene encodes a truncated MYBS2 transcription factor which includes deletion of 1st-53rd amino acid residues.

14. The method according to claim 11, wherein the plant is a monocotyledonous plant or a dicotyledonous plant.

15. The method according to claim 14, wherein the monocotyledonous plant is selected from the group consisting of Zea mays, Sorghum bicolor, Setaria italica, Hordeum vulgare, Brachypodium distachyon, Oryza sativa, Triticum spp., and Saccharum spp.

16. The method according to claim 14, wherein the dicotyledonous plant is selected from the group consisting of Cucumis sativus, Ricinus communis, Solanum lycopersicum, Solanum tuberosum, Vitis vinifera, Populus trichocarpa, Arabidopsis thaliana, Arabidopsis lyrata, and Platycodon grandiflorus.

17. The method according to claim 11, wherein the plant is a crop.

18. The method according to claim 17, wherein the crop is rice, maize, wheat, barley, sugarcane, banana, cotton, soybean, pea, potato, tomato, brassica, orchid, balloon flower, yam, sweet potato, cassava, rose, petunia, chrysanthemum, lily, or carnation.

19. The method according to claim 11, wherein the plant is an angiosperm.

20. The method according to claim 11, wherein the abiotic stress factor is osmotic stress, salt, dehydration, or heat.


SEQUENCE LISTING (22 sequences)

SEQ ID NO: 1 - DNA, 25 bp, Artificial Sequence
  synthetic primer
gtaggcaggctctctagcctctagg

SEQ ID NO: 2 - DNA, 23 bp, Artificial Sequence
  synthetic primer
aacctgacattatatattgcacc

SEQ ID NO: 3 - DNA, 26 bp, Artificial Sequence
  synthetic primer
ctcagggttcctgccggtagaaagca

SEQ ID NO: 4 - DNA, 19 bp, Artificial Sequence
  synthetic primer
cgaaacgaacagtagctag

SEQ ID NO: 5 - DNA, 20 bp, Artificial Sequence
  synthetic primer
cagaccaaccctggcaaaaa

SEQ ID NO: 6 - DNA, 23 bp, Artificial Sequence
  synthetic primer
gaggacttggaagctgatcatca

SEQ ID NO: 7 - DNA, 21 bp, Artificial Sequence
  synthetic primer
accacttcgaccgccactact

SEQ ID NO: 8 - DNA, 19 bp, Artificial Sequence
  synthetic primer
acgcctaagcctgctggtt

SEQ ID NO: 9 - DNA, 33 bp, Artificial Sequence
  synthetic primer
gcgcggccgcaccatggagcagcatgaggaggc

SEQ ID NO: 10 - DNA, 30 bp, Artificial Sequence
  synthetic primer
gcagatcttcacagcaccttgatggccgcc

SEQ ID NO: 11 - DNA, 29 bp, Artificial Sequence
  synthetic primer
gcgcggccgcatgcccaacctcacctcca

SEQ ID NO: 12 - DNA, 25 bp, Artificial Sequence
  synthetic primer
gcagatctcagcaccttgatggccg

SEQ ID NO: 13 - DNA, 1144 bp, Oryza sativa
agtctcctccaaaccgtcgagagcccaacaataataatccccccttttcatcgcggcgacgatacccgccattttcatctcattctcaccaccaaaagcgaccacgcagcgaccggccaccgatcgagatggagcagcatgaggaggcagcggagaggaagccttcgccgccggtgatattccggctgttcggcgtcgaggtccgcggcggcggcggcggagttgacgaggaggagtacgaggaggaggaggtggagggtggattgttcatcaagaagagctccagtatgcccaacctcacctccatcgacccgctgccggtgccggccgacggcggcaaacggcgcgcctccgacgactccgagctcgcctccggccagcagaagcgccgccgccgcaaggtgcaggagaggaagaaaggggtaccatggactgaggaggagcacaagaaattcctggaagggctgaggcagctggggaaaggggactggagaggcatctccaagaactttgtgaccagcaggacggcgactcaggtggccagccacgcccagaagtacttcctccggcagaccaaccctggcaaaaagaagcgccgggccagcctctttgatgttgttgctgagtgcagtgatgatcagcttccaagtcctcagagtgttggaactaagcctcctacccaggatataattcatacagatcgcggcgatgtcccgatactaagctatccagttgctagaggctttagaggcgatagcgtgcaggttgatgaactaactgaatatgtgaagagattaaaggccgccgaggacatgtcgctctccatgatctctggactggaaatggcatcatcatccatcagcagtctagagctcagtatcgcgcctcctcattgcgcgatcgaggcggccatcaaggtgctgtgatccagacgcaattctccaccgaaggaatctggatcggcttcagctactgttcttcgtcgccgctgttgttgtttgttgttgttgttttttttttttttttgcgggggttgtttgttgttgttgttgttgtagttgtcatgctaactttgtatttgggtcatgtggcgtttctttcagcagtcttatataatacagtgagaatgtcagtaccttccgag

SEQ ID NO: 14 - DNA, 639 bp, Artificial Sequence
  cDNA
atgcccaacctcacctccatcgacccgctgccggtgccggccgacggcggcaaacggcgcgcctccgacgactccgagctcgcctccggccagcagaagcgccgccgccgcaaggtgcaggagaggaagaaaggggtaccatggactgaggaggagcacaagaaattcctggaagggctgaggcagctggggaaaggggactggagaggcatctccaagaactttgtgaccagcaggacggcgactcaggtggccagccacgcccagaagtacttcctccggcagaccaaccctggcaaaaagaagcgccgggccagcctctttgatgttgttgctgagtgcagtgatgatcagcttccaagtcctcagagtgttggaactaagcctcctacccaggatataattcatacagatcgcggcgatgtcccgatactaagctatccagttgctagaggctttagaggcgatagcgtgcaggttgatgaactaactgaatatgtgaagagattaaaggccgccgaggacatgtcgctctccatgatctctggactggaaatggcatcatcatccatcagcagtctagagctcagtatcgcgcctcctcattgcgcgatcgaggcggccatcaaggtgctgtga

SEQ ID NO: 15 - DNA, 485 bp, Artificial Sequence
  MYBS2RNAI----3`UTR 485bp
tccagacgcaattctccaccgaaggaatctggatcggcttcagctactgttcttcgtcgccgctgttgttgtttgttgttgttgttttttttttttttttgcgggggttgtttgttgttgttgttgttgtagttgtcatgctaactttgtatttgggtcatgtggcgtttctttcagcagtcttatataatacagtgagaatgtcagtaccttccgagacatgtttaattaagccttggttggttggtataagttggtgaaaagaaaaccagatagtagcctgtctgtattgtacggttgcaaaattggagtatattgatgcagagatcatgaagggtctccatcttgttcataagtctagtttgtttcctgttcctgtcacaggaggcagctgggacatcttcttgtgtggctctgagcttcttccagtgcttcaataattggacccttccccttcatgtccttgtgaccattggtcccagcca

SEQ ID NO: 16 - DNA, 28 bp, Artificial Sequence
  Guide RNA sequence of MYBS2 CRISPR/Cas9 lines
ggcataatctgtgaaggggtaccagttt

SEQ ID NO: 17 - DNA, 1986 bp, Oryza sativa
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgca

SEQ ID NO: 18 - DNA, 588 bp, Oryza sativa
ccgcggtgtcgtttcccgccttcagtttagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgacgcggccgcacc

SEQ ID NO: 19 - DNA, 256 bp, Oryza sativa
ggatcccccgggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatc

SEQ ID NO: 20 - DNA, 1314 bp, Oryza sativa
gtgcgagatccaccacccgatgacctcccaggcggcgacgacgacgaccacggcggcggcggcggcggcgtggaccagggaggacgacaaggcgttcgagaacgcgctcgcggcgtgcgcggcgccgccgcccgcggacggaggcgcgcccgacgacgactggttcgccgcgctcgccgcgagcgtgcccggggcgaggtcggcggaggaggtgcggaggcactacgaggcgctggtggaggacgtcgcggccatcgacgcgggccgcgtcccgctcccgcgctacgccggggaggagtccgcggcgccgcccgacggagccggagccgccgccgccgcgtccaaggacggcggacaccggcgcgacgagcgcaagggcggcggcggcgggtacgacggcggcaagagctgctccaaggcggagcaggagaggcgcaagggcatcccatggacggaggaagagcacaggctgttcttgctggggctggacaagttcggcaagggggactggcggagcatctcgcgcaacttcgtcatctcgcggacgccaacgcaggtggcgagccacgcgcagaagtacttcatccgcctcaactccatgaaccgcgaccgccgccgctccagcatccacgacatcaccagcgtcaccgccggcgatcaggtcgccgcgcagcagggcgccccgatcaccggccaccaggccacgggcaaccccgcggcggcggcgctgggcccgccgggcatgaagcaccaccaccaccaccacccgggcggcgcgccgccgcccatgcccatgtacagcgccgcgcccatgggccaccccgtcgccggccacatggtgcccgccgccgtcggcacgccggtggtgttcccgccgggccacgcgccgtacgtcgtgcccgtcggctacccggcgccaccggccaagatgcaccaatgacgcgccatggacggacatgagcagcatttcttcctcctcctttcttgatgtgaatcttgatttgttctttgtgtagtcgccggctcatcgtccctgatcatcttgttcttctcacaatctcactaatgtaaacatacatagatcagatgccaagagtgcagggattggggattaaaggcgaataagtaaagtattttgctgactgtttgcaagtgatcatcacgtacacccggtgaaagcttagctccaaatgtggatgtaattagcagcggccttccgtacgtggtggcgccgatcgatgatcttgcaggggttgcaattagggattgatttccattttgctgatgtaaatttgcctactgtctcattggacc

SEQ ID NO: 21 - DNA, 1773 bp, Oryza sativa
attcccctgccacggtgaccgtgcccccggtgttctatatatgccccccgacgtcgaggtcattcgccacgaacacatcgatcatccatcatctacaagagatcgatcagtagtggttagcagcaactcactatcgaacacggtttcagcttacacagatatgaagaacaccagcagcttgtgtttgctgctcctcgtggtgctctgcagcttgacctgtaactcgggtcaagcacaggtcctcttccagggtttcaactgggagtcgtggaagcagcagggtggctggtacaacatgttgaaaggccaagtcgacgacatcgccaaggccggggtcacccacgtctggctgccgccgccgtcgcactccgtggcgccgcaggggtacatgccggggcgtctctacgacctggacgcgtccaagtacggcacggcggcggagctcaagtcgctgatcgcggcgttccacgggaagggcgtccagtgcgtcgccgacgtcgtgatcaaccaccggtgcgccgagaagaaggacgcccgcggcgtgtactgcgtgttcgagggcgggacgcccgacgaccgcctcgactggggccccggcatgatctgcagcgacgacacgcagtactccgacggcacgggccaccgcgacaccggcgaggggttcggcgcggcgcccgacatcgaccacctcaacccgcgcgtccagcgggagctcaccgactggctcaactggctcaagtccgacgtcggcttcgacggctggcgcctcgacttcgccaagggatactccacggacatcgctaagatgtacgtcgagagctgcaagccgggcttcgtcgtcgccgagatatggaactcgctgagctacaacggcgacggcaagccggcggccaaccaggaccagggccggcaggagctggtgaactgggtgaacgccgtcggcgggccggcgatgacgttcgacttcaccaccaagggcctcctgcaggcgggcgtccagggcgagctgtggcggctgcgcgacggcaacggcaaggccgccggcatgatcgggtggctgccagagaaggccgtcacgttcgtcgacaaccacgacaccggctcgacgcagaagctttggccgttcccctccgacaaggtcatgcagggctacgcctacatcctcacccaccccggagtcccctgcatcttctacgaccacatgttcgactggaacctgaagcaggagataaccgcgctggcggcgatcagggagaggaacggcatcaacgccgggagcaagctccggatcgtcgtcgccgacgccgacgcatacgtcgccgtcgtcgacgagaaggtcatggtgaagatcgggacgaggtacgacgtgggcaacgcggtgccgtcggatttccatcagacggtgcacggcaaggactacagcgtctgggagaaggggtccctccgcgtcccggcggggcggcacctatagcgggctcaagccctaaactgaacgggatagtcatgctcaaaccagtttctacacggcaagaatttactgattcttatacttttgcagtcaattaaattatggtttttatatatgtaattttgtatccgattgtagcgttcgaataagtaggcaggctctctagcctctaggttaattgcggggcatatgtagcttgccagttaattgtgtttgtatcacgcagtttgtaaccgttggtgcaatatataatgtcaggttcaggatgcagtaaaaaatcatactgcaccgatcagtgagttttt

SEQ ID NO: 22 - protein, 265 aa, Oryza sativa
MEQHEEAAERKPSPPVIFRLFGVEVRGGGGGVDEEEYEEEEVEGGLFIKKSSSMPNLTSIDPLPVPADGGKRRASDDSELASGQQKRRRRKVQERKKGVPWTEEEHKKFLEGLRQLGKGDWRGISKNFVTSRTATQVASHAQKYFLRQTNPGKKKRRASLFDVVAECSDDQLPSPQSVGTKPPTQDIIHTDRGDVPILSYPVARGFRGDSVQVDELTEYVKRLKAAEDMSLSMISGLEMASSSISSLELSIAPPHCAIEAAIKVL